(19) United States
(12) Patent Application Publication
(10) Pub. No.: US 2024/0101719 A1
(43) Pub. Date: Mar. 28, 2024

(54) YEAST GLUCANS, METHODS AND USES THEREOF

(71) Applicants: Universidade Catolica Portuguesa-UCP, Porto (PT); Amyris Bio Products Portugal, Unipessoal LDA, Porto (PT)

(72) Inventors: Joao Cruz Fernandes, Porto (PT); Maria Manuela Faria Amorim, Matosinhos (PT); Pedro Miguel Constante De Sousa, Maia (PT); Diana Maria Tavares Valente, Arouca (PT); Joao Pedro Azevedo Silva, Trofa (PT); Christopher J. Paddon, Pacifica, CA (US); Maria Manuela Estevez Pintado, Gondomar (PT)

(21) Appl. No.: 18/276,761

(22) PCT Filed: Feb. 11, 2022
(86) PCT No.: PCT/IB2022/000067

§ 371 Date: Aug. 10, 2023

(30) Foreign Application Priority Data
Feb. 14, 2021 (PT) 117062

(51) Int. Cl. C08B 37/00; A61K 39/39; A61P 37/04

(52) U.S. Cl. CPC C08B 37/0003; A61K 39/39; A61P 37/04; C08B 37/0009; C08B 37/0024; A61K 2039/55583

(57) ABSTRACT

The present disclosure relates to method of obtaining high purity glucans, in particular yeast glucans with immunomodulatmy properties, including vaccine adjuvant properties for the use as adjuvant in vaccines.

### TECHNICAL FIELD

The present disclosure relates to method of obtaining high purity yeast glucans for the use as a vaccine adjuvant.

### BACKGROUND

Glucans, prevalent among Saccharomyces cerevisiae cell wall, are complex polysaccharides consisting of repeated units of d-glucose linked by a mixture of glycosidic bonds—β-(1-3); (1-6) and α-(1,4)-D—glucan. Isolation of glucans from yeasts has been well documented and explored in the form of β-glucans that possess the ability of enhancing and stimulating the human immune system and have been proven beneficial for various human and animal diseases and disorders. On the other hand, there is a lack of information on the bioactivity of yeast cell wall α-(1,4)-glucans as an isolated fraction.

The biological properties of glucans are known, and include antibacterial, antiviral, tissue regeneration, anti-herpetic, and immunostimulatory activities such as pro-inflammatory effects, and as vaccines adjuvants. Glucans act as non-self-molecules, namely pathogen-associated molecular patterns (PAMPs). Glucan are recognized by pattern recognition receptors (PRRs) which are expressed by many different immune cells. The interaction between β-glucans and their receptor activates different signalling cascades. For instance, through binding to dectin-1, β-(1,3)-(1,6), glucans activate innate immune responses such as phagocytosis, reactive oxygen species (ROS) production and inflammatory cytokines production in macrophages.

To date it has neither been technically possible nor economically feasible to synthesise glucan from yeast cell on a commercial basis. The foregoing facts are disclosed strictly to illustrate the technical problem addressed by the present disclosure.

### GENERAL DESCRIPTION

The present disclosure relates to method of obtaining high purity yeast glucans with immunomodulatory properties, including vaccine adjuvant properties.

As aspect of the present disclosure relates to a method of obtaining yeast glucan extract wherein the quantity of peptide, namely peptide contaminants, in the extract is not more than 0.1% (wt/wt), the method comprising the following steps:
obtaining autolyzed yeasts cells;
extract dried alkali-glucans by adding an alkaline solution to the yeast cells in a solid-liquid extraction and collecting a first pellet of the first solid-liquid extraction;
adding to the obtained first pellet of the first extraction an alcoholic solution and an acid solution in a second solid-liquid extraction to obtain a second pellet of the second solid-liquid extraction;
adding a suitable detergent to the obtained second pellet to obtain a deproteinized glucan extract.

In an embodiment, the method of the present disclosure may further comprise spray drying the deproteinized glucan extract before purifying the deproteinized glucan extract.

In an embodiment, the method of the present disclosure may further comprise spray functionalizing the purified glucan extract with carboxymethyl groups.

In an embodiment, the yeast cell may be Saccharomyces cerevisiae; Pichia pastoris, Cyberlindnera jadinii, Candida albicans, or mixtures thereof. In a preferred embodiment, the yeast glucans may be extracted from spent yeast from S. cerevisiae. Preferably Saccharomyces strain CEN.PK2 Genotype: MATa/α ura3-52/ura3-52 trp1-289/trp1-289 leu2-3,112/leu2-3,112 his3 Δ1/his3 Δ1 MAL2-8C/MAL2-8C SUC2/SUC2, CEN.PK possesses a mutation in CYR1 (A5627T corresponding to a K1876M substitution near the end of the catalytic domain in adenylate cyclase which eliminates glucose- and acidification-induced cAMP signaling and delays glucose-induced loss of stress resistance).

In an embodiment, adding to 0.5-2 g of dried alkali-glucans 60-120 ml of an alcoholic solution and 1-3 ml of an acid solution; preferably to 1 g of dried alkali-glucans 80 ml of an alcoholic solution and 1.6 ml of an acid solution.

In an embodiment, the first solid-liquid extraction is performed in an orbital shaker.

In an embodiment the first solid-liquid extraction is performed during 3-6 hours at a temperature ranging from 45-60° C., preferably 5 hours at 50° C.

In an embodiment, the alkaline solution is sodium hydroxide solution, preferably 0.5-2 M.

In an embodiment, the acid solution may be hydrochloric acid solution, preferably 5-10 M.

In an embodiment, the method of the present disclosure may further comprise a step of washing the pellet; preferably multiples washing with ethanol and acetone.

Method according to any of the previous claims comprising adding 80-150 ml of detergent to 0.5-2 g of the obtained solid glucan extract, preferably 100 ml of detergent to 1 g of the obtained solid glucan extract.

In an embodiment, the alcoholic solution may be an ethanol solution, preferably 96-99% (v/v).

In an embodiment, the detergent may be selected from a list consisting of sodium dodecyl sulphate, Tween 20, Triton, and mixtures thereof.

In an embodiment, the present disclosure relates to an extraction process for obtaining highly pure glucan fractions from spent yeast, wherein the resulting glucan extract comprises a low concentration of peptide contaminants, in particular, not more than 0.1% (wt/wt).

In an embodiment, the present disclosure relates to an extraction process for obtaining highly pure glucan fractions from yeast, wherein the resulting glucan extract comprises vaccine adjuvant properties.

In an embodiment, two chemical extractions and purification approaches were carried out with immunomodulatory properties, including vaccine adjuvant properties.

In an embodiment, the extracted yeast glucans may be further functionalized by addition of a carboxymethyl group. This functionalization allowed to improve the glucans water solubility and improved performance as vaccine adjuvant.

In an embodiment, the extracted yeast glucans may be further functionalized by addition of a water-soluble molecule, including but not limited to, a sulphate, an acetate or a carboxymethyl group. This functionalization allowed to improve the glucans water solubility and improved performance as vaccine adjuvant.

Another aspect of the present disclosure relates to extracted glucans obtainable by the method described in the present disclosure wherein the quantity of peptide contaminants in the deproteinized glucan extract is not more than 0.1% (wt/wt), preferably not more than 0.05% (wt/wt), more preferably not more than 0.018% (wt/wt).

In the present disclosure, the following peptides are considered a peptide contaminant in the deproteinized glucan extract: Mannoproteins linked to β-1,6- glucose chains through a processed glycosylphosphatidylinositol (GPI) anchor or to β-1,3-glucan through an alkali-labile bond.

In an embodiment, glucans obtainable by the method described in the present disclosure may be use in medicine or as a medicament. Namely, as a carrier in the treatment or therapy of viral infection.

In an embodiment, the glucans described in the present disclosure may be use in the prevention or treatment of viral infections. Namely as a lead adjuvant in a vaccine, namely a lead adjuvant.

Another aspect of the present disclosure relates to pharmaceutical composition comprising the extracted glucan described in the present disclosure.

In an embodiment, the composition may be administrated as a nasal spray, as an intravenous preparation.

Another aspect of the present disclosure relates to the use of the glucans obtained by the method described in the present disclosure as a vaccine adjuvant or immune stimulant. In particular, a lead adjuvant.

In an embodiment, the vaccine is a prophylactic vaccine.

In another embodiment, the vaccine is a SARS-CoV-2 vaccine.

Another aspect of the present disclosure relates to the use of the glucans obtained by the method described in the present disclosure in therapies against infectious agents.

Another aspect of the present disclosure relates to the use of the glucans obtained by the method described in the present disclosure in therapies against intracellular pathogens.

In an embodiment, In particular, the intracellular pathogen is a virus.

Another aspect of the present disclosure relates to the use of the glucans obtained by the method described in the present disclosure as a vaccine adjuvant, where the glucans are combined with aluminum salts.

Another aspect of the present disclosure relates to the use of the glucans obtained by the method described in the present disclosure as a vaccine adjuvant, where the glucans are combined with squalene.

Another aspect of the present disclosure relates to the use of the glucans obtained by the method described in the present disclosure as a vaccine adjuvant, where the glucan further comprises a carboxymethyl group and the glucans are combined with aluminum salts.

Another aspect of the present disclosure relates to the use of the glucans obtained by the method described in the present disclosure as a vaccine adjuvant, where the glucan further comprises a carboxymethyl group and the glucans are combined with squalene.

Another aspect of the present disclosure relates to pharmaceutical composition comprising the extracted glucan described in the present disclosure and at least one of aluminum salts and squalene.

Another aspect of the present disclosure relates to the use of the glucans obtained by the method described in the present disclosure as a COVID-19 vaccine adjuvant.

### BRIEF DESCRIPTION OF THE DRAWINGS

The following figures provide preferred embodiments for illustrating the disclosure and should not be seen as limiting the scope of invention.

FIG. 1 is a schematic representation of the process for extraction of different glucans.

FIG. 2 shows the THP-1 cells viability determined by propidium iodide staining exposed with glucans for 24 hours.

FIGS. 3a to 3c show the concentrations of cytokines (IL-8, IL-6 and TNF-α) in THP-1 cells that have been exposed to glucans for 24 hours (normalized to total protein).

FIG. 4 shows Cytokines and Chemokines production levels for PBMCs isolated from 8 donors and exposed to glucans (0.05 mg/ml) combined with Alum or SE.

FIG. 5 shows levels of Antigen-specific IgG producing splenocytes in mice immunized with SE and glucans formulations. Black dots correspond to females and white dots to males.

FIG. 6 shows levels of antigen-specific IgG producing splenocytes in mice immunized with Alum and glucans formulations, where black dots correspond to females and white dots to males.

FIG. 7 shows levels of antigen-specific IgG producing splenocytes in mice immunized with the different adjuvants used independently, where black dots correspond to females and white dots to males.

FIG. 8 shows antigen-specific titer in mice immunized with SE formulations., where black dots correspond to females and white dots to males.

FIG. 9 shows antigen-specific titer in mice immunized with Alum formulations, where black dots correspond to females and white dots to males.

FIG. 10 shows antigen-specific titer in mice immunized with the different adjuvants alone, where black dots correspond to females and white dots to males.

FIG. 11 shows different classes/subclasses of immunoglobulins observed in different groups immunized with SE formulations.

FIG. 12 shows different classes/subclasses of immunoglobulins observed in different groups immunized with Alum formulations.

FIG. 13 shows cytokines expression in different groups immunized with SE formulations.

FIG. 14 shows cytokines expression in different groups immunized with Alum formulations.

FIG. 15 shows pseudovirus spike protein neutralization assay for mice immunized with different formulations, where black dots correspond to females and white dots to males.

### List of Abbreviations

Glu-RbM—Glucan Alkali+Organic/acid treatment from RebM spent yeast;
Pure-RbM—Glucan Alkali+Organic/acid+SDS treatment from RebM spent yeast;
CM GIu-RbM—Carboxymethyl Glucan from RebM spent yeast;
Glu-WT—Glucan Alkali+Organic/acid treatment from wild type yeast CEN.PK2;
Pure-WT—Glucan Alkali+Organic/acid+SDS treatment from wild type yeast CEN.PK2;
CM Glu-WT—Carboxymethyl Glucan from wild type yeast CEN.PK2.

### DETAILED DESCRIPTION

The present disclosure relates to method of obtaining high purity yeast glucans with antiviral properties and vaccines adjuvant properties. The present disclosure further relates to method of obtaining high purity glucans, in particular yeast glucans with vaccine adjuvant properties.

In an embodiment, the present disclosure relates to an extraction process for obtaining highly pure glucan fractions from at least one of spent and fresh yeast, wherein the resulting glucan extract comprises a low concentration of peptide contaminants, in particular, not more than 0.1% (wt/wt) of peptides contaminants.

In an embodiment, the present disclosure relates to an extraction process for obtaining highly pure glucan fractions from spent yeast, wherein the resulting glucan extract comprises vaccine adjuvant properties.

In an embodiment, yeast glucans were extracted from spent yeast from the production of sweetener molecule. These strains may be genetically modified organism (GMO), glucans from the wild-type Saccharomyces strain CEN.PK2 was also extracted. Saccharomyces strain CEN.PK2 Genotype: MATa/α ura3-52/ura3-52 trp1-289/trp1-289 leu2-3,112/leu2-3,112 his3 Δ1/his3 Δ1 MAL2-8C/MAL2-8C SUC2/SUC2, CEN.PK possesses a mutation in CYR1 (A5627T corresponding to a K1876M substitution near the end of the catalytic domain in adenylate cyclase which eliminates glucose- and acidification-induced cAMP signaling and delays glucose-induced loss of stress resistance.

In an embodiment, two chemical extractions and purification approaches were carried out with vaccine adjuvants as target.

In an embodiment, the extracted yeast glucans were further functionalized by addition of carboxymethyl groups.

In an embodiment, the yeast glucans were extracted via alkali and organic acid treatment.

In an embodiment, the yeasts were subjected to heat treatment in order to release cellular components—autolysis, enabling the isolation and purification of the glucans present in the insoluble fraction (pellet) of the yeast cell wall polysaccharides. After autolysis, the first step in the extraction process was initiated by an alkaline treatment, where an initial 20% (w/v) solution was prepared using autolyzed yeast pellet and sodium hydroxide (NaOH 1 M) as a solvent. Thereafter, this solution was placed in a water bath at 90° C. for 2-4 hours. After this, it was centrifuged at 8000 rpm for 10 min at 4° C. and the supernatant was then discarded. The resulting pellet was washed three times by centrifugation with deionized water. The washed pellet was re-suspended in 50 mL of deionized water and neutralized with hydrochloric acid (HCl 3 M) until pH 7 was obtained. The supernatant was removed by centrifugation and a last wash was done using the same volume of deionized water. The resulting pellet, containing insoluble alkali-glucans, was completely homogenized in deionized water and dried by spray drying, at an inlet temperature of 110° C. and an outlet of 50° C.

In an embodiment, in order to achieve higher purity glucan content, any remaining proteins, lipids and other unwanted compounds were removed using acid/ethanol extraction. For this extraction, 1 g of dried alkali-glucans was dissolved in 80 ml of ethanol (99%) and 1.6 ml of HCl 32% (w/v). The mixture of yeast glucans and solvent (ethanol and HCl) was placed in an orbital shaker for 4 hours at 50° C. Thereafter, the pellet was washed three times—twice with absolute ethanol and once with acetone—suspending 20 ml of each solvent and centrifuging at 4° C., for 10 min, at 8000 rpm. Purified glucans were dried overnight in a vacuum oven at 50° C.

In an embodiment, the glucans extracted were characterized in terms of their physicochemical composition—protein, lipids, moisture, minerals (see Table 1 below).

In an embodiment, the extracted yeast glucans were further functionalized.

In an embodiment, the addition of functional groups to glucans improves its water solubility, bioactivities, and increases its biocompatibility.

In an embodiment, the yeast glucans were functionalized with the addition of carboxymethyl groups (CM). The functionalization was performed using monochloroacetic acid and alkali treatment of the alkali and organic acid treated glucans obtained earlier from either RebM or CEN.PK2.

In an embodiment, the yeast glucans were purified via SDS (sodium dodecyl sulphate) treatment to obtain purified yeast glucans for use as a vaccine adjuvant. Optionally, prior to SDS treatment, glucans can be further purified by enzymatic digestion, with enzymes such as Promozyme at 1% at pH 6 and 60° C. overnight.

In an embodiment, a SDS wash at high temperatures was performed to remove the small quantity of residual proteinaceous still present in the glucans extract. 1 g of glucans extracted through alkali and organic acid treatment was mixed with 100 mL of SDS at 2% (w/v) and placed in a water bath at boiling temperature for 15 min. Thereafter, a centrifugation at 8000 rpm, at 4° C. for 10 min was done and the supernatant discarded. Thereafter, the sample was diluted again in 100 mL of SDS and placed in boiling water again. After centrifugation, this process was repeated one more time. After three boils, the pellet was washed three times with deionized water and two times with absolute acetone; the supernatants were discarded in each wash by centrifuging the sample. Immediately before drying, 1 mL of acetone was added to the remaining pellet and it was homogenized to help the drying process. Purified glucans were dried overnight in a vacuum oven at 50° C. Optionally, as noted above, prior to SDS treatment steps disclosed, glucans can be further purified by enzymatic digestion, with enzymes such as Promozyme at 1% at pH 6 and 60° C. overnight.

In an embodiment, the purified yeast glucan sample was characterized in terms of its physicochemical composition—protein, lipids, moisture, minerals (see Table 1 below).

TABLE 1
Chemical composition of each glucan extract.

| | | % dw | | | | |
|---|---|---|---|---|---|---|
| Extraction process | Strain | Moisture | Minerals | Protein | Carbohydrates | Lipids |
| Glucan | RbM | 2.0 ± 0.1 | 1.1 ± 0.3 | 3.9 ± 0.1 | 90.9 ± 3.8 | 1.0 ± 0.1 |
| Alkali + Organic/ | CEN.PK2 | 1.0 ± 0.1 | 0.8 ± 0.3 | 2.0 ± 0.1 | 94.9 ± 3.8 | 0.5 ± 0.1 |
| acid treatment | | | | | | |
| Glucan | RbM | 1.0 ± 0.1 | <0.01 | <0.01 | 99.0 ± 1.1 | n.d |
| Alkali + Organic/ | CEN.PK2 | 0.5 ± 0.3 | <0.01 | <0.01 | 99.6 ± 3.8 | n.d |
| acid + SDS treatment | | | | | | |

n.d—not detected

In an embodiment, ß-glucan content was determined by enzymatic procedure for the measurement of 1,3-1,6-β-glucan in yeast (assay kit Megazyme). The content in different yeast glucan extracts were analyzed and the results set out in Table 2 below. Glucans extracted by different processes have a mixture of type ß and α—linkages, with ß links in greater percentage.

TABLE 2
β-glucan content of each fraction

% dw

| Extraction process | Strain | β-Glucan | α-Glucan |
|---|---|---|---|
| Glucan Alkali + Organic/ | RbM | 59.0 ± 0.2 | 31.8 ± 1.2 |
| acid treatment | CEN.PK2 | 64.6 ± 0.4 | 30.3 ± 0.2 |
| Glucan Alkali + Organic/ | RbM | 64.0 ± 2.2 | 35.6 ± 2.2 |
| acid + SDS treatment | CEN.PK2 | 70.0 ± 0.4 | 29.3 ± 1.0 |

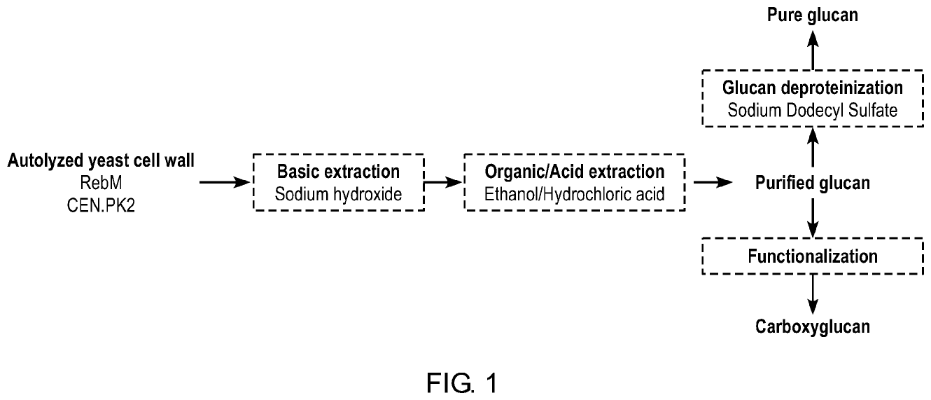

FIG. 1 is a schematic representation of the extraction process of different glucans.

The beneficial effects of ß-glucans as immunomodulators are extensively studied. As described herein, they have the capacity to trigger immunologic responses by interacting with specific macrophages receptors.

The potent immunostimulatory activity of glucans has been studied concerning the vaccine adjuvants development. As described herein, different methodologies to extract glucans, with a high purity degree, without residual proteinaceous content (<0.1%) important for activation of immune cells and cytokine production stimulation have been developed.

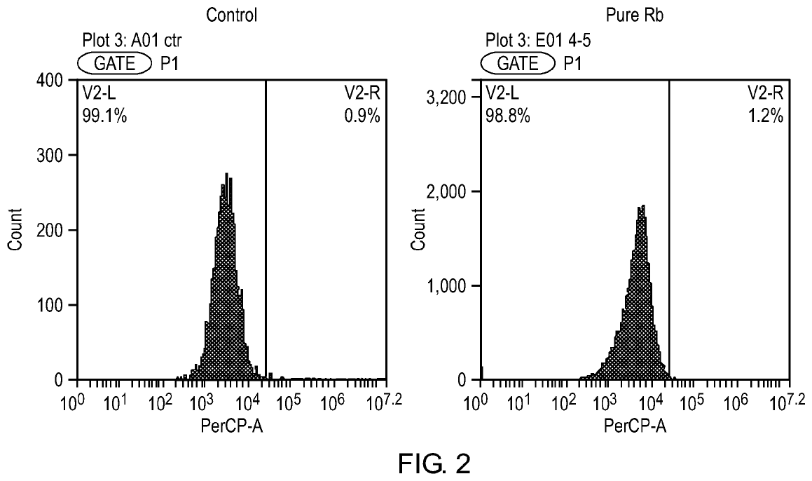

In the embodiment, the cytotoxicity of pure glucans (Pure-RbM and Pure-WT) in monocytes-(THP-1 cells) was evaluated by flow cytometry using propidium iodide (12.5 μg/mL). The results show that pure glucans were not cytotoxic, a selected population of THP-1 cells demonstrating 98.8% of viability (FIG. 2).

FIG. 2 shows the THP-1 cells viability determined by propidium iodide staining exposed to glucans for 24 hours.

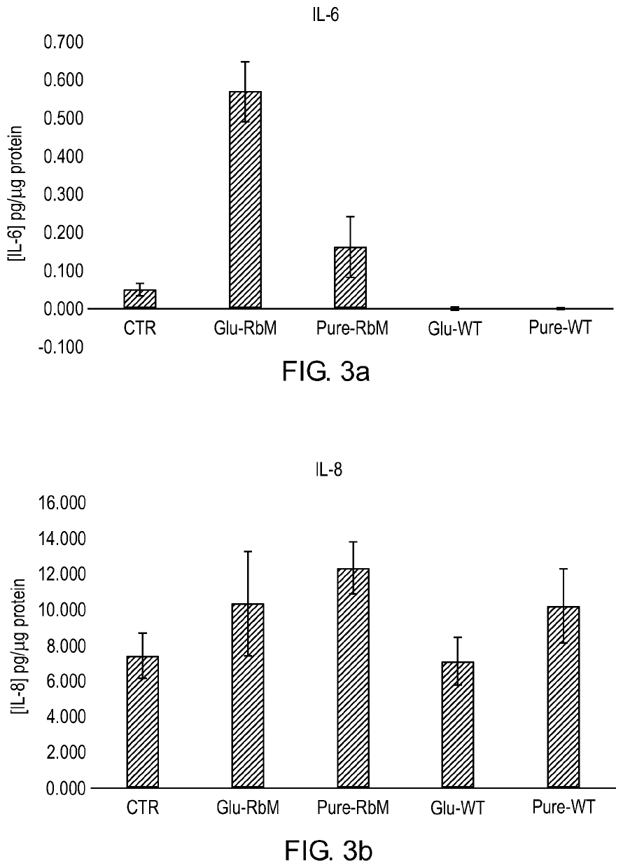

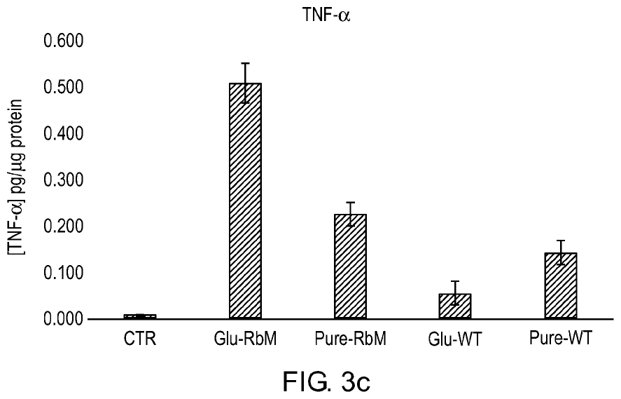

In an embodiment, the immunomodulatory activity of the extracted yeast glucans was evaluated by ELISA. THP-1 cells were seeded in 24-well plates at 3.5×10⁵ cells/ml and exposed to phorbol-12-myristate-13-acetate (PMA, 50 nM) for 48 hours in order to induce macrophage differentiation. Thereafter, the cells were exposed to different glucans for 24 hours. The supernatants were collected and used for cytokines quantification (Elisa Max Deluxe Set Human, Biolegend) following manufacturer's instructions. Pure glucans induced a pro-inflammatory response as demonstrated by an increase in IL-8, IL-6 and TNF-α production as compared with control (Table 3 and FIGS. 3a to 3c), except Pure-WT IL-6 production.

FIGS. 3a to 3c show the concentrations of cytokines (IL-8, IL-6 and TNF-α) in THP-1 cells that have been exposed to glucans for 24 hours (normalized to total protein).

TABLE 3
Production of cytokines in THP-1 cells (relative
to control) exposed to glucans for 24 hours.

| | Relative percentage of production (to control) | | |
|---|---|---|---|
| Samples | IL-8 | IL-6 | TNF-α |
| CTR | 100.0 | 100.0 | 100.0 |
| Glu-RbM | 139.3 | 1125.3 | 6534.0 |
| Pure-RbM | 166.2 | 318.6 | 2904.6 |
| Glu-WT | 95.8 | n.d. | 708.8 |
| Pure-WT | 137.6 | n.d. | 1843.4 |

n.d—not detected

Table 3 shows the capacity of β-glucans to increase the expression of inflammatory cytokines such as Il-8, IL-6 and TNF-α is a proof of its immunomodulatory properties. This demonstrates ability of the use of β-glucans of the present disclosure as a vaccine adjuvant.

### β-Glucans Adjuvants Embodiments

To evaluate the properties of highly pure glucans as vaccine adjuvants or co-adjuvants, the following embodiments have been developed in combination with aluminum salts and squalene. While not described in the tables below, combinations of glucans with other vaccine adjuvants are herein disclosed. The concentrations and different combinations tested are presented in Table 4.

TABLE 4
Glucans formulations for efficacy testing (in vitro)

| | | Glucans | SE | Alum |
|---|---|---|---|---|
| Description | | (mg/mL) | (%) | (μg/mL) |
| Saline | | — | — | — |
| Aluminum (Alum) | | — | — | 20, 40, |
| | | | | 80 e 160 |
| Squalene (SE) | | — | 0.05, 0.1, | — |
| | | | 0.2 e 0.4 | |
| Glucans wild-type | — | 0.05, 0.1, | — | — |
| carboxymethylated | c/Alum | 0.2 e 0.4 | — | 20, 40, |
| | | | | 80 e 160 |
| | c/SE | | 0.05, 0.1, | — |
| | | | 0.2 e 0.4 | |
| Glucans wild-type | — | 0.05, 0.1, | — | — |
| | c/Alum | 0.2 e 0.4 | — | 20, 40, |
| | | | | 80 e 160 |
| | c/SE | | 0.05, 0.1, | — |
| | | | 0.2 e 0.4 | |
| Glucans RebM | — | 0.05, 0.1, | — | — |
| carboximethylated | c/Alum | 0.2 e 0.4 | — | 20, 40, |
| | | | | 80 e 160 |
| | c/SE | | 0.05, 0.1, | — |
| | | | 0.2 e 0.4 | |
| Glucans RebM | — | 0.05, 0.1, | — | — |
| | c/Alum | 0.2 e 0.4 | — | 20, 40, |
| | | | | 80 e 160 |
| | c/SE | | 0.05, 0.1, | — |
| | | | 0.2 e 0.4 | |
| Glucans RebM | — | 0.05, 0.1, | — | — |
| Promozyme | c/Alum | 0.2 e 0.4 | — | 20, 40, |
| | | | | 80 e 160 |
| | c/SE | | 0.05, 0.1, | — |
| | | | 0.2 e 0.4 | |

### Chemokines and Cytokines Production: In Vitro Analysis

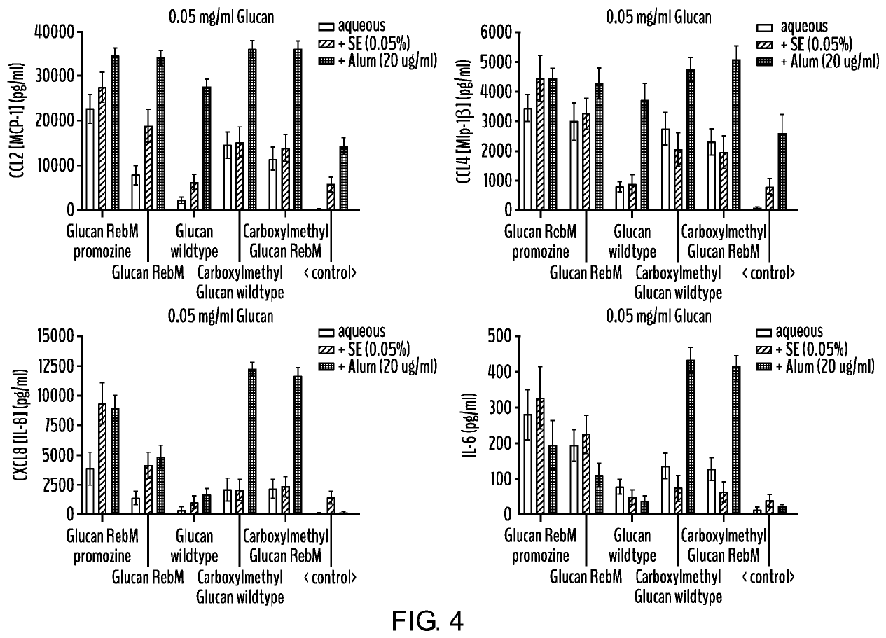

As disclosed herein, embodiments utilizing formulations of glucans were used to evaluate cytokine and chemokine production in human peripheral blood mononuclear cells (PBMCs). For example, according to one embodiment, blood from 8 individuals (4 women and 4 men) were collected and PBMCs isolated. Then, cells were exposed to glucans alone or formulated with aluminum or squalene and, after 24 h, cells were centrifuged and the serum was used for the evaluation of IL-6, IL-8, MCP-1 and Mip-1β levels by ELISA. The results, shown in FIG. 4, shown that for lower concentrations (0.05 mg/ml), glucans RebM seem to induce a higher immunologic response than other glucans alone. Surprisingly, when combined with Alum, glucans show a significative better performance acting synergistically with Alum, especially for carboxymethylated glucans. Mixtures of glucans with squalene lead to modest chemokines production levels.

The aforementioned results demonstrate the immunomodulatory abilities of the different glucans tested, whereby soluble glucans (carboxymethylated) have better performance when formulated with aluminum and, glucans RebM hold better performance when formulated with squalene.

### Glucans Embodiments for Stability and In Vivo Testing

Embodiments with higher concentrations of glucans, Alum or SE were used to evaluate formulation stability and antigen interaction to develop a vaccine candidate for COVID-19. Mixtures were performed according to Table 5 and the stability of the embodiments was evaluated after 4 h at 5° C. and 25° C. Visual analysis (Table 6) concluded that for color, opacity and phase, no alteration occurred for the designated timepoints.

The pH and temperatures of each sample were analyzed using a pH PerpHecT Ross Combination pH microeletrode and a pH Accumet AB150, respectively. The pH and temperature values are presented in Table 7. No significative alterations were observed after 4 h for these parameters.

Particle dimension was analyzed by Dynamic Light Scattering (DLS). In Table 8, only formulations of SE were analyzed as in formulations of Alum particle size was below the detection limit. In SE embodiments no alteration in particle size was detected while in formulations of glucans with antigen was observed a significant variation in particle size and in the polydispersity index (see Table 8)

TABLE 5
Glucans embodiments for stability and in vivo testing-

| Description | | Supplier | Details | code |
|---|---|---|---|---|
| Saline | | Ricca | 0.9% NaCl | 1909D70 |
| Spike S1 Protein | | Genescript | 1.67 mg/mL | P50132006 |
| Alum | | IDRI | 2 mg/mL Alum | QH863 |
| SE | | IDRI | 5% SE | QH864 |
| Glucans | — | IDRI | 0.5 mg/mL | QH865 |
| wild-type | | | glucans | |
| carboxymethylated | w/Alum | IDRI | 0.5 mg/mL | QH866 |
| | | | glucans + | |
| | | | 2 mg/mL Alum | |
| | w/SE | IDRI | 0.5 mg/mL | QH867 |
| | | | glucans + | |
| | | | 5% SE | |
| Glucans | — | IDRI | 0.5 mg/mL | QH868 |
| wild-type | | | glucans | |
| | w/Alum | IDRI | 0.5 mg/mL | QH869 |
| | | | glucans + | |
| | | | 2 mg/mL Alum | |
| | w/SE | IDRI | 0.5 mg/mL | QH870 |
| | | | glucans + | |
| | | | 5% SE | |
| Glucans | — | IDRI | 0.5 mg/mL | QH871 |
| RebM | | | glucans | |
| | c/SE | IDRI | 0.5 mg/mL | QH872 |
| | | | glucans + | |
| | | | 5% de SE | |
| Glucans | — | IDRI | 0.5 mg/mL | QH873 |
| RebM | | | glucans | |
| Promozyme | c/SE | IDRI | 0.5 mg/mL | QH874 |
| | | | glucans + | |
| | | | 5% de SE | |

TABLE 6
Visual evaluation of the tested glucan embodiments

| | | | T = 4 h | T = 4 h |
|---|---|---|---|---|
| Group | | T = 0 h | 5° C. | 25° C. |
| Antigen | | Clear, no color, | No | No |
| | | one phase | alterations | alterations |
| Alum | | White, translucid, | No | No |
| | | one phase | alterations | alterations |
| SE | | White, opaque, | No | No |
| | | one phase | alterations | alterations |
| Glucans | — | Clear, no color, | No | No |
| wild-type | | one phase | alterations | alterations |
| carboxymethylated | w/Alum | White, translucid, | No | No |
| | | one phase | alterations | alterations |
| | w/SE | White, opaque, | No | No |
| | | one phase | alterations | alterations |
| Glucans | — | Clear, no color, | No | No |
| wild-type | | one phase | alterations | alterations |
| | w/Alum | White, translucid, | No | No |
| | | one phase | alterations | alterations |
| | w/SE | White, opaque, | No | No |
| | | one phase | alterations | alterations |
| Glucans | — | Clear, no color, | No | No |
| RebM | | one phase | alterations | alterations |
| | w/SE | White, opaque, | No | No |
| | | one phase | alterations | alterations |
| Glucans | — | Clear, no color, | No | No |
| RebM | | one phase | alterations | alterations |
| Promozyme | w/SE | White, opaque, | No | No |
| | | one phase | alterations | alterations |

TABLE 7
Formulation pH analysis

| Formulation | | T = 0 h | T = 4 h 5° C. | T = 4 h 25° C. |
|---|---|---|---|---|
| Antigen | | 7.16 | 7.22 | 7.42 |
| Alum | | 6.85 | 6.76 | 6.59 |
| SE | | 6.04 | 6.05 | 6.05 |
| Glucans wild-type | — | 7.58 | 7.24 | 7.62 |
| carboxymethylated | w/Alum | 6.69 | 6.54 | 6.68 |
| | w/SE | 5.94 | 6.08 | 6.03 |
| Glucans wild-type | — | 7.44 | 7.19 | 7.00 |
| | w/Alum | 6.65 | 6.49 | 6.47 |
| | w/SE | 6.02 | 6.04 | 6.09 |
| Glucans RebM | — | 7.36 | 7.00 | 7.36 |
| | w/SE | 6.04 | 6.01 | 6.08 |
| Glucans RebM | — | 7.26 | 6.94 | 7.27 |
| Promozyme | w/SE | 6.17 | 6.06 | 6.06 |

TABLE 8
Particle dimension analysis

| | | T = 0 h | | T = 4 h 5° C. | | T = 4 h 25° C. | |
|---|---|---|---|---|---|---|---|
| Group | | Size (d · nm) | PDI | Size (d · nm) | PDI | Size (d · nm) | PDI |
| SE | | 74.3 ± 1.59 | 0.048 ± 0.012 | 74.3 ± 1.67 | 0.050 ± 0.010 | 74.7 ± 2.20 | 0.042 ± 0.017 |
| Glucans wild-type | — | 706 ± 487 | 0.683 ± 0.177 | 608 ± 136 | 0.682 ± 0.212 | 857 ± 486 | 0.670 ± 0.246 |
| carboxymethylated | w/SE | 75.3 ± 1.73 | 0.054 ± 0.016 | 75.8 ± 1.70 | 0.049 ± 0.018 | 75.2 ± 1.89 | 0.051 ± 0.018 |
| Glucans wild-type | — | 3847 ± 1745 | 1.00 ± 0.000 | 3326 ± 414 | 1.000 ± 0.000 | 6956 ± 6087 | 0.941 ± 0.156 |
| | w/SE | 74.4 ± 1.83 | 0.057 ± 0.011 | 74.8 ± 1.73 | 0.049 ± 0.015 | 74.5 ± 2.14 | 0.046 ± 0.012 |
| Glucans RebM | — | 1997 ± 1526 | 0.886 ± 0.080 | 5376 ± 8328 | 0.975 ± 0.075 | 6290 ± 10470 | 0.920 ± 0.237 |
| | w/SE | 74.4 ± 1.81 | 0.053 ± 0.021 | 74.2 ± 1.01 | .052 ± 0.015 | 073.9 ± 1.31 | 0.054 ± 0.012 |
| Glucans RebM | — | 7789 ± 10860 | 0.922 ± 0.100 | 3374 ± 1265 | 0.946 ± 0.163 | 5454 ± 8007 | 0.920 ± 0.155 |
| Promozyme | w/SE | 74.5 ± 1.75 | 0.051 ± 0.009 | 74.4 ± 1.45 | 0.050 ± 0.010 | 74.3 ± 1.77 | 0.043 ± 0.011 |

Antigen adsorption in mixtures was also evaluated. In an embodiment, the mixtures with Alum were incubated 5 min at room temperature and centrifuged for 10 min at 21000 g. The presence of virus-like particles in the supernatants was evaluated by SDS-PAGE. No alteration in Spike S1 protein was observed in SE and aqueous formulations. Moreover, antigen was not detected in supernatant of Alum formulations, indicating that antigen was completely absorbed by Alum.

Taking into account the foregoing results, the formulations are shown to be stable for at least 4 h at 5° C. and 25° C. being the antigen well adsorb in the Alum formulations. The foregoing results further indicate a good compatibility of the Spike S1 SARS-CoV-2 protein with all the adjuvants/formulations tested. All the studied formulations were used for in vivo tests.

### Evaluation of the Efficacy Glucans Embodiments as Adjuvants in Covid-19 Vaccines

In an embodiment, the immunomodulatory potential of glucans formulations was evaluated in vivo in mice C57BL/6J (Mus musculus). The glucans concentration used was low in order to better assess the glucans immunomodulatory capacity. According to conducted experiments, the results of which are shown below, 140 mice were immunized, distributed by 15 different groups (5 males and 5 females per group) as presented in Table 9. Animals were immunized twice, at the beginning of the experiment (day 0) and at day 21. After 3 weeks of the second immunization, animals were sacrificed and the blood serum was collected to further evaluation of antibody titers, antibodies subclasses (IgG1, IgG2a, IgG2b, IgG3, IgA and IgM), chemokines (CCL2/CCL3/CCL4/CCL5/CCL17/CCL22/CXCL10), cytokines (IFN-Y, IL-1, IL-4, IL-6, IL-10, IL-13, IL-17A, IL-22, IL-12 (p70)) and neutralization efficiency of antibodies against SARS-COV-2 pseudovirus. Splenocytes from both mice femurs were isolated and analyzed by ELISPOT to evaluate their capacity to produce immunoglobulins specific for the antigen used in the experiment.

TABLE 9
Group of animals for the immunization.

| Group | | | Feeding | Administration |
|---|---|---|---|---|
| 1 | Saline | | Food and | 25 + 25 μL |
| | | | water ad | saline solution |
| | | | libitum | (0.9% NaCl) |
| 2 | Spike S1 | | | 25 + 25 μL antigen |
| | Protein | | | (10 μg total) |
| 3 | Alum | | | 25 + 25 μL |
| | | | | Alum at 2 mg/mL |
| 4 | SE | | | 25 + 25 μL |
| | | | | SE at 5% |
| 5 | Glucans | — | | 25 + 25 μL |
| | wild-type | | | glucans at 0.5 mg/mL |
| 6 | carboxymethylated | w/Alum | | 25 + 25 μL glucans |
| | | | | 0.5 mg/mL + |
| | | | | Alum at 2 mg/mL |
| 7 | | w/SE | | 25 + 25 μL glucans |
| | | | | at 0.5 mg/mL + |
| | | | | SE at 5% |
| 8 | Glucans | — | | 25 + 25 μL glucans |
| | wild-type | | | at 0.5 mg/mL |
| 9 | | w/Alum | | 25 + 25 μL glucans |
| | | | | 0.5 mg/mL + |
| | | | | Alum at 2 mg/mL |
| 10 | | w/SE | | 25 + 25 μL glucans |
| | | | | at 0.5 mg/mL + |
| | | | | SE at 5% |
| 11 | Glucans | — | | 25 + 25 μL glucans |
| | RebM | | | at 0.5 mg/mL |
| 12 | | w/SE | | 25 + 25 μL glucans |
| | | | | at 0.5 mg/mL + |
| | | | | SE at 5% |
| 13 | Glucans | — | | 25 + 25 μL glucans |
| | RebM | | | at 0.5 mg/mL |
| 14 | Promozyme | w/SE | | 25 + 25 μL glucans |
| | | | | at 0.5 mg/mL + |
| | | | | SE at 5% |

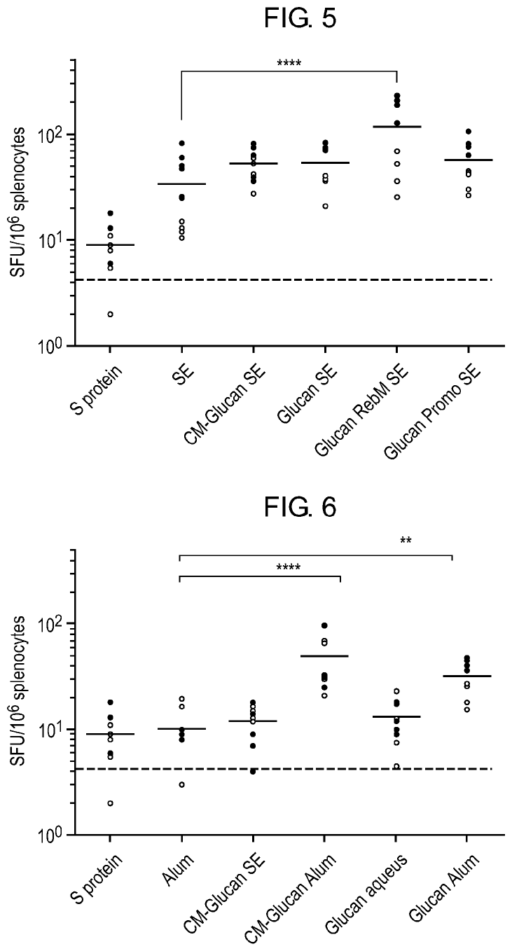

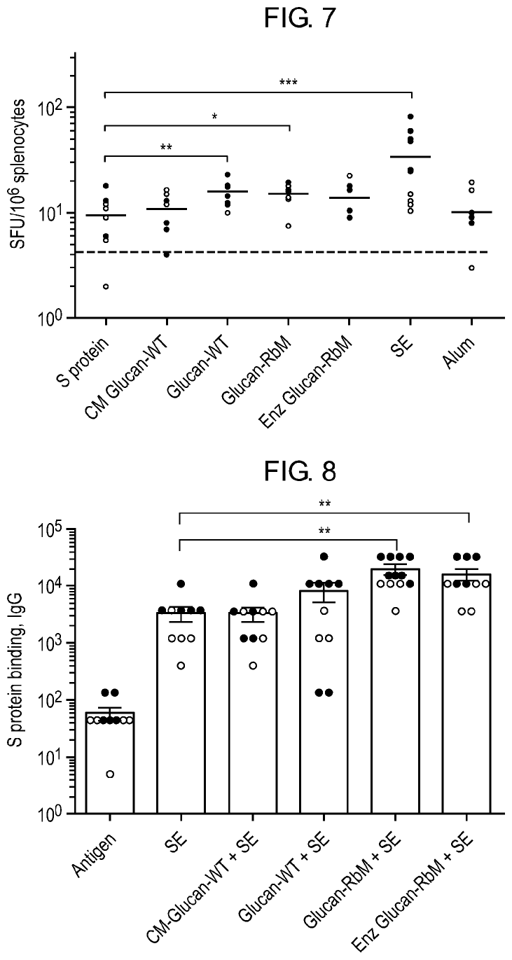

The development of an immunity response the spike S1 protein was evaluated by ELISPOT. Splenocytes were isolated from both femurs, purified from red blood cells and the number of cells capable of produce antigen-specific IgG was quantified; the results were expressed as spot forming units (SFU)/10⁶ splenocytes (FIGS. 5-7). In FIGS. 5-7, the following symbols are used: * P<0.05; ** P<0.01; *** P<0.001; **** P<0.0001 by Wilcoxon non-parametric test. The impact of the formulations was higher in females than males. Nevertheless, the foregoing results clearly show that combinations of Alum or SE with glucans outperform the use of these classic adjuvants alone. The combination of glucans RebM with SE lead to a significant increase in the number of splenocytes producers of antigen-specific IgG (average of mice immunized with antigen=9.45; average antigen+SE=30.91; average antigen+SE+glucans RebM=117.38). The combination of SE with other glucans also lead to an increase in antigen-specific producer cells however to a lower extent (average antigen+SE+glucans wild type carboxymethylated=53.35; average antigen+SE+glucans RebM Promozyme=57.11).

As disclosed herein, embodiments combining Alum with glucans also resulted in a stimulation of the immunologic response (average antigen+Alum=10.2; average antigen+Alum+glucans wild-type carboxymethylated=50.0; antigen+Alum+glucans wild-type=31.88).

The use of isolated glucans as adjuvants resulted in similar values obtained by the Alum alone, with non-carboxymethylated glucans presenting higher values (average antigen+glucans wild-type=15.89; antigen+glucans RebM=15.19; antigen+glucans RebM Promozyme=14.06). Nevertheless, these values are lower than SE alone (30.91).

The results obtained by ELISPOT showed that combining glucans with other adjuvants such as SE or Alum result in a significant increase in the number of splenocytes producing antigen-specific IgG (2-5× times higher). SE combined better with glucans RebM while Alum showed a higher increase when combined with soluble (carboxymethylated) glucans.

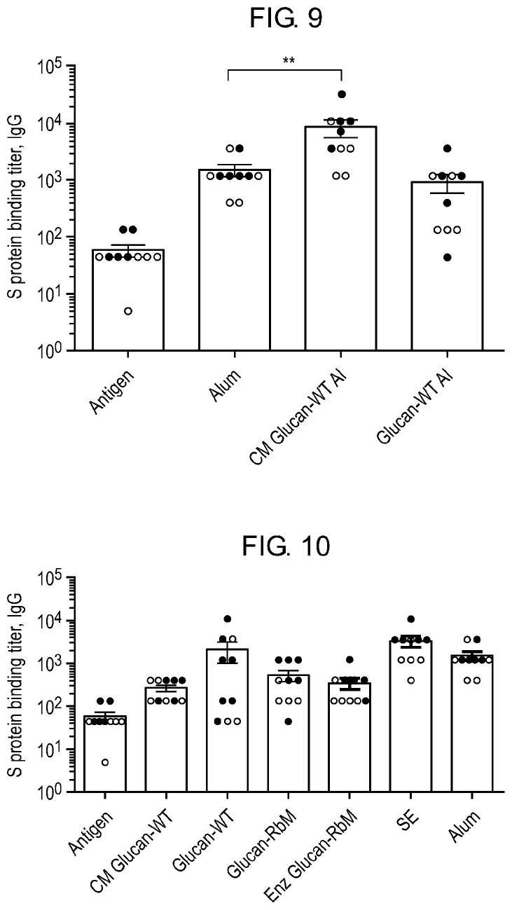

The titters of antigen-specific IgGs in the blood serum was determined by ELISA (FIGS. 8-10). In FIGS. 8-10, the following symbols are used: * P<0.05; ** P<0.01; *** P<0.001; **** P<0.0001 by one-way ANOVA. The results obtained are aligned with the results from ELISPOT. Embodiments combining SE with glucans RebM significantly increase antigen-specific IgG titters (SE=3321; SE+glucans wild-type=8289; SE+glucans RebM=19845; SE+glucans RebM Promozyme=16038). The combination of glucans carboxymethylated with Alum also lead to an increase in IgG titter in relation to the use of Alum alone (Alum=11305; Alum+glucans wild-type carboxymethylated=83770; Alum+glucans wild-type=1025). When used as sole adjuvants, glucans lead to an increase in IgG titters similarly to Alum or SE being significantly higher than the use of the antigen alone. No difference was observed between the different glucans tested.

The results obtained demonstrate the potential of glucans RebM to promote antigen-specific IgG production when combined with SE (5× higher than SE alone). Aligned also with the results from ELISPOT, Alum embodiments with soluble glucans presented a better result than native glucans (5× higher than antigen alone).

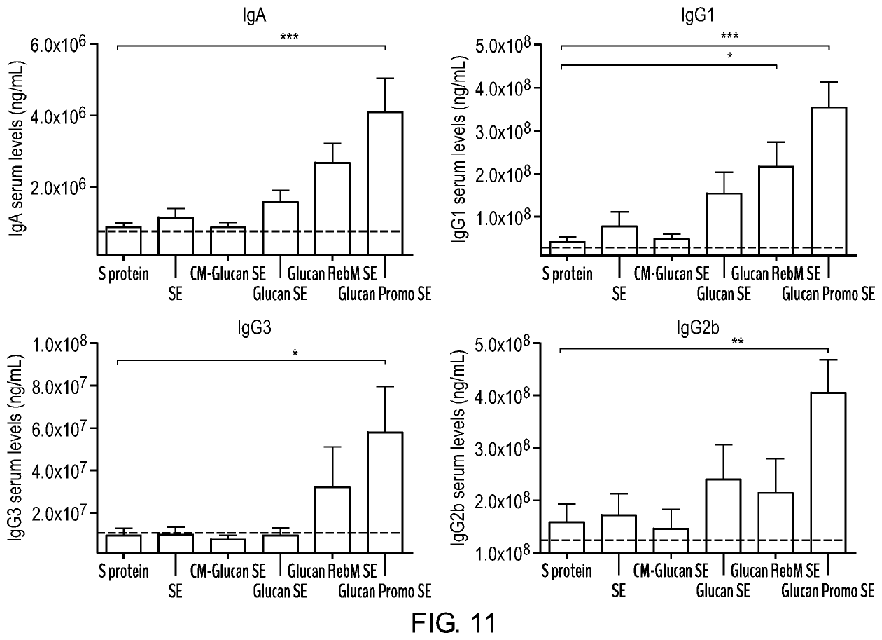

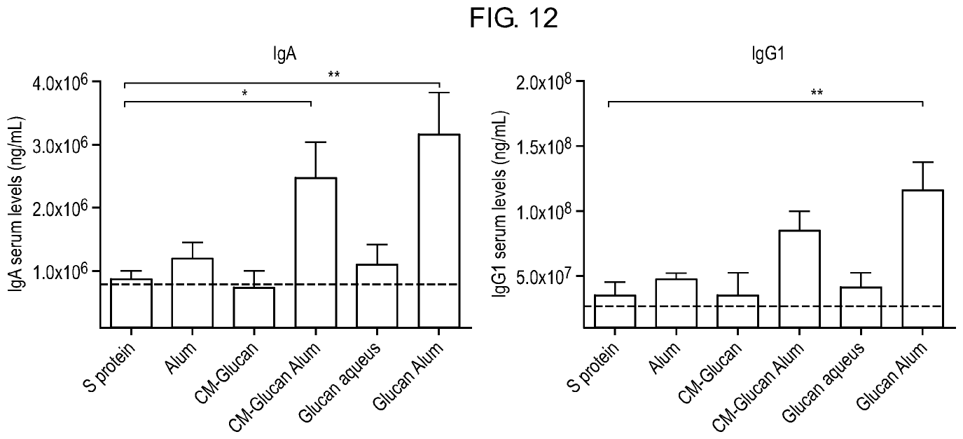

The evaluation of antibody titers, antibodies subclasses (IgG1, IgG2a, IgG2b, IgG3, IgA and IgM) was performed using a multiplex by flow cytometry (FIGS. 11 and 12). In FIGS. 11 and 12, the presented values correspond to the group average. * P<0.05; ** P<0.01; *** P<0.001; **** P<0.0001 by Wilcoxon non-parametric test. For SE embodiments, differences were observed for IgA, IgG1, IgG2a and IgG3 (FIG. 11). Although, only for IgG1 these differences were significative when comparing naïve and mice immunized with SE+glucans RebM. For Alum embodiments, differences were lower than with SE embodiments. Significative increase in immunoglobulins levels were observed for IgA (Alum+glucan wild-type and Alum+glucan wild-type carboxymethylated) and IgG1 (Alum+glucan wild-type).

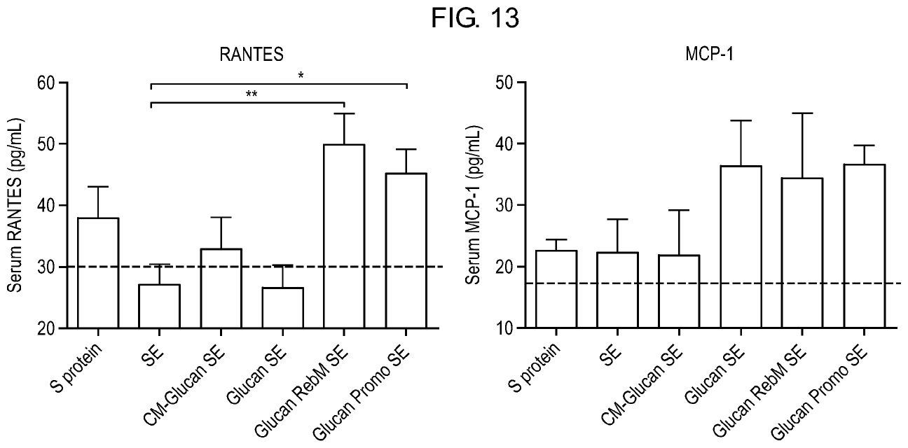

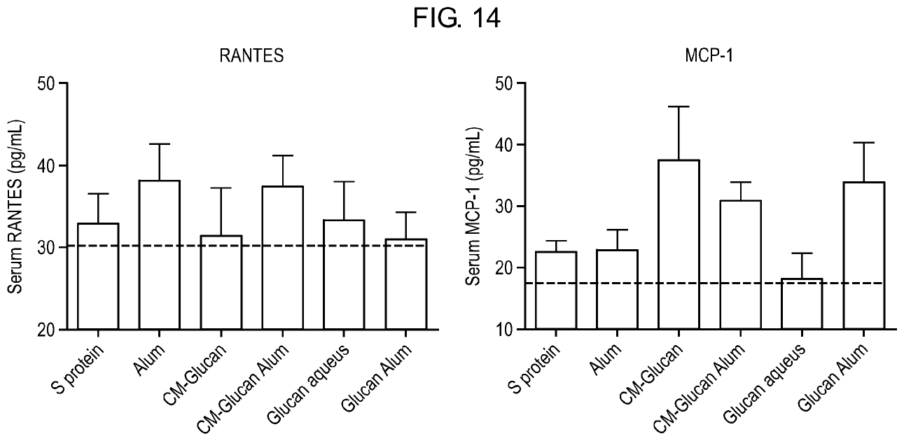

Cytokines and chemokines expression in the blood serum were evaluated using a multiplex analysis by flow cytometry. From the panel analyzed (RANTES, TARC, MCP-1, IP10, MIP1α, MIP-1β and MDC), it was detected slight differences only for RANTES (CCL5) and MCP-1 (CCL2), namely for SE embodiments in comparison to physiological levels (FIG. 13). Nevertheless, the differences observed for SE combined with glucans RebM were not statistically significative in comparison to mice immunized only with the antigen. For Alum embodiments the results were also the same with no statistically differences observed for the different embodiments tested (FIG. 14). In FIG. 14, the presented values correspond to the group average and the following symbols are used: * P<0.05; ** P<0.01; *** P<0.001; **** P<0.0001 by one-way ANOVA.

Regarding the data shown in FIGS. 13 and 14, it is speculated that the reason behind the lack of significant alterations in chemokines and cytokines is due to the fact that mice were sacrificed 3 weeks after the last immunization. This time window was enough to enable reestablishment of the physiological levels which might have been altered right upon immunization.

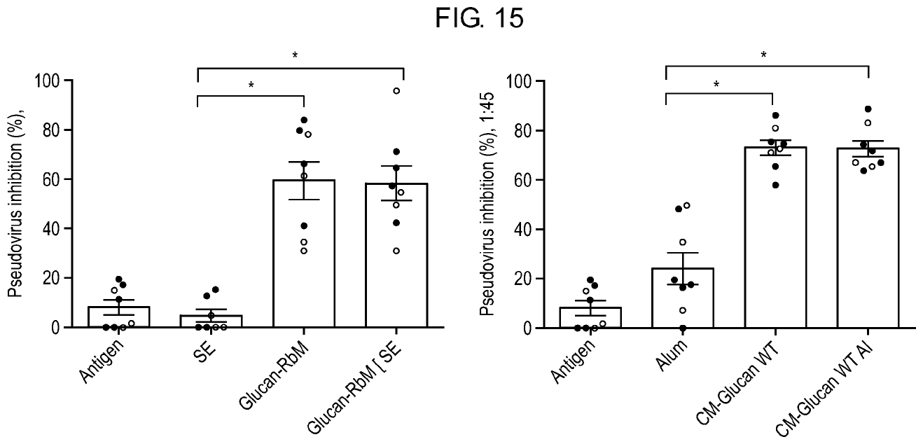

To evaluate the capacity of the immunized mice to neutralize the interaction of SARS-CoV-2 with its receptor ACE-2 present on the surface of human cells, a pseudovirus methodology was implemented. For example, a pseudovirus (not replicative) expressing SARS-CoV-2 spike protein was used which, upon entering human cells lead to the expression of a green fluorescence protein (GFP) and a HEK-293 cell line modified to express ACE2 receptor. Cells were infected with the pseudovirus alone or previously incubated with a serial dilution of blood serum from immunized mice. The presence of GFP positive cells were analyzed by flow cytometry and the results expressed as percentage of pseudovirus inhibition (FIG. 15). In FIG. 15, the presented values correspond to each mice in its respective group and the group average, and the following symbols are used: * P<0.05; ** P<0.01; *** P<0.001; **** P<0.0001 by Wilcoxon non-parametric test. The results indicate that mice immunized with glucans RebM and glucans carboxymethylated wild-type (alone or in combination with SE or Alum, respectively) were able to neutralize the antigen, preventing the binding to ACE2 receptor. This inhibition was significantly higher than in mice immunized with SE or Alum alone.

The foregoing results clearly demonstrate the uses and embodiments of glucans as vaccine adjuvants, especially when combined with other adjuvants such especially, glucans RebM combined with SE and soluble glucans (wild-type carboxymethylated) with Alum. These embodiments enable to significantly enhance the immune response against the presence of the antigen; for example, the Spike S1 protein from SARS-COV-2. This immune response is translated by an increase in the number of splenocytes able to produce antigen-specific IgGs, as well as, by an increase in the level of these immunoglobulins in the circulatory system and their capacity to neutralize the pseudovirus expressing SARS-CoV-2 spike protein on its surface. Moreover, the embodiments disclosed herein increase the levels of IgA, a class of immunoglobulins associated with mucosa protection.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof.

The embodiments described above are combinable.

What is claimed is:

1. Method of obtaining yeast glucan extract wherein the quantity of peptide, namely peptide contaminants, in the extract is not more than 0.1% (wt/wt), the method comprising the following steps:
obtaining autolyzed yeasts cells;
extract dried alkali-glucans of the yeast by adding an alkaline solution to the yeast cells in a solid-liquid extraction and collecting a first pellet;
adding to the obtained first pellet an alcoholic solution and an acid solution in a second solid-liquid extraction to obtain a second pellet;
adding a suitable detergent to the obtained second pellet to obtain a deproteinized glucan extract.

2. Method according to any of the previous claims further comprising spray drying the deproteinized glucan extract before purifying the deproteinized glucan extract.

3. Method according to any of the previous claims further comprising functionalizing the purified glucan extract with carboxymethyl groups.

4. Method according to the previous claim wherein the yeast cell is Saccharomyces cerevisiae; Pichia pastoris, Cyberlindnera jadinii, Candida albicans, or mixtures thereof.

5. Method according to any of the previous claims comprising adding to 0.5-2 g of dried alkali-glucans 60-120 ml of an alcoholic solution and 1-3 ml of an acid solution; preferably to 1 g of dried alkali-glucans 80 ml of an alcoholic solution and 1.6 ml of an acid solution.

6. Method according to any of the previous claims wherein the first solid-liquid extraction is performed during 3-6 hours at a temperature ranging from 45-60° C., preferably 5 hours at 50° C.

7. Method according to any of the previous claims wherein the alkaline solution is sodium hydroxide, preferably 0.5-2 M.

8. Method according to any of the previous claims wherein the acid solution is hydrochloric acid solution, preferably 5-10 M.

9. Method according to any of the previous claims further comprising a step of washing the first pellet; preferably multiples washing with ethanol and acetone.

10. Method according to any of the previous claims comprising adding 80-150 ml of detergent to 0.5-2 g of the obtained solid glucan extract, preferably 100 ml of detergent to 1 g of the obtained solid glucan extract.

11. Method according to any of the previous claims wherein the alcoholic solution is an ethanol solution, preferably 96-99% (v/v).

12. Method according to any of the previous claims wherein the detergent is selected from a list consisting of sodium dodecyl sulphate, Tween 20, Triton, and mixtures thereof; preferably sodium dodecyl sulphate, more preferably sodium dodecyl sulphate 2% (w/v).

13. Extracted glucans obtainable by the method according to any of the previous claims wherein the quantity of peptide contaminants in the deproteinized glucan extract is not more than 0.1% (wt/wt), preferably not more than 0.05% (wt/wt), more preferably not more than 0.018% (wt/wt).

14. Extracted glucans obtainable according to the previous claim wherein the glucan further comprises a carboxymethyl group.

15. Extracted glucans obtainable according to any of the previous claims for use in medicine or as a medicament.

16. Extracted glucans obtainable according to any of the previous claims for use as a carrier in the treatment or therapy of viral infection.

17. Extracted glucans obtainable according to any of the previous claims for use in the prevention or treatment of viral infections.

18. Pharmaceutical composition comprising the extracted glucan described in the claims 14-18.

19. Pharmaceutical composition according to the previous claim, wherein the composition is administrated as a nasal spray, as an intravenous preparation.

20. Use of the glucans obtained by the method described in any of the previous claims as a vaccine adjuvant or immune stimulant.

21. Use of the glucans according to the previous claim wherein the adjuvant is a lead adjuvant.

22. Use of the glucans according to the previous claim wherein the vaccine is a prophylactic vaccine.

23. Use of the glucans according to the previous claim wherein the vaccine is a COVID-19 vaccine.

24. Extracted glucans obtainable according to any of the previous claims for use as a vaccine adjuvant, wherein in the glucans are combined with aluminum salts.

25. Extracted glucans obtainable according to any of the previous claims for use as a vaccine adjuvant, wherein in the glucans are combined with squalene.

26. Extracted glucans obtainable according to claim 1 for use as a vaccine adjuvant, wherein the glucan further comprises a carboxymethyl group and the glucan is combined with aluminum salts.

27. Extracted glucans obtainable according to claim 1 for use as a vaccine adjuvant, wherein the glucan further comprises a carboxymethyl group and the glucan is combined with squalene.

28. Pharmaceutical composition comprising the extracted glucan described in the claims 14-18 and at least one of aluminum salts and squalene.

29. Use of the glucans obtained by the method described in any of the previous claims as a COVID-19 vaccine adjuvant.

30. Use of the glucans obtained by the method described in any of the previous claims as a therapy against infectious agents.

31. Use of the glucans according to the previous claim wherein the infectious agent is a intracellular pathogens.

32. Use of the glucans according to the previous claim wherein the intracellular pathogen is a virus.

33. Use of the glucans obtained by the method described in any of the previous claims wherein the glucan further comprises a water-soluble molecule.

34. Use of the glucans according to the previous claim wherein water-soluble molecule comprises at least one of a sulphate, an acetate and a carboxymethyl group

35. Extracted glucans obtainable according to claim 1 for use as a therapy against infectious agents, wherein the glucan further comprises a water-soluble molecule.

36. Extracted glucans obtainable according to any of the previous claims for use as a vaccine adjuvant, wherein in the glucans are combined with aluminum salts.

37. Extracted glucans obtainable according to any of the previous claims for use as a vaccine adjuvant, wherein in the glucans are combined with squalene.
